(19) United States
(12) Patent Application Publication
(10) Pub. No.: US 2024/0228429 A1
(43) Pub. Date: Jul. 11, 2024

(54) PROCESS FOR PRODUCING L-CARNITINE

(71) Applicants: HUBEI XUJIE PHARMACEUTICAL CO., LTD, Jingmen city (CN); CARNITECH LLC, North Brunswick, NJ (US)

(72) Inventors: Songzhou Hu, Princeton, NJ (US); Zhen Song, Shanghai (CN)

(21) Appl. No.: 18/093,164

(22) Filed: Jan. 4, 2023

(51) Int. Cl. C07C 227/18

(52) U.S. Cl. CPC C07C 227/18

(57) ABSTRACT

There is disclosed an improved process for the production of L-carnitine, comprising the steps of: (a) mixing an organic base with an acid solution comprised of L-carnitine to form an organic base salt of the acid, optionally in the presence of a solvent; (b) removing the organic base salt of step (a) to yield a solution comprised of L-carnitine; and (c) isolating the L-carnitine from the solution of step (b).

### TECHNICAL FIELD

The present invention relates to an improved process for the production of L-carnitine, in particular, it relates to a process for reducing the formation of inorganic salts in a solution of L-carnitine.

### BACKGROUND OF THE INVENTION

L-carnitine is a naturally occurring quaternary ammonium acid involved in metabolism in most mammals, plants, and some bacteria. L-carnitine is also called vitamin BT and has a molecular structure of formula (I). It plays a critical role in energy production by transporting long-chain fatty acids into mitochondria so they can be oxidized to produce energy. L-carnitine also plays an important role in the regulation of metabolic pathways involved in skeletal muscle protein balance. Furthermore, L-carnitine acts as an anti-oxidant and as an anti-inflammatory compound. Therefore, L-carnitine finds wide applications as a nutritional supplement and feed additive.

There are many methods for the synthesis of L-carnitine, but only two processes are being used commercially.

The first process is the fermentative oxidation of gamma-butyrobetaine as depicted in the following reaction:

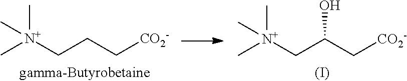

The second process starts from epichlorohydrin and accounts for the majority of L-carnitine produced. The reactions in the second process are described in the following scheme:

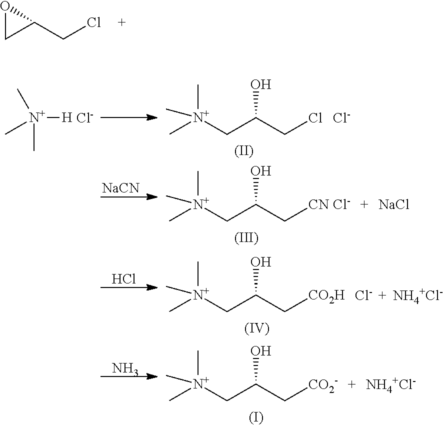

Racemic epichlorohydrin can be efficiently resolved into (S)-epichlorohydrin by using a Jacobsen Co(Salen) catalyst in high yield and high optical purity. (S)-epichlorohydrin is then reacted with trimethylamine hydrochloride to form L-3-chloro-2-hydroxypropyl trimethylammonium chloride of formula (II), which is subsequently reacted with sodium cyanide to form L-carnitinenitrile chloride of formula (III). These two steps can be carried out in a one-pot process without isolating the intermediate (II) in water. After isolation and purification, L-carnitinenitrile chloride is hydrolyzed in concentrated hydrochloric acid to yield L-carnitine and ammonium chloride. L-carnitine is finally isolated from this solution comprised of L-carnitine, excess hydrochloric acid, and ammonium chloride. The process according to this reaction scheme has been described in U.S. Pat. No. 9,096,493.

The reaction of epichlorohydrin with trimethylamine hydrochloride to form 3-chloro-2-hydroxyltrimethylammonium chloride is well known. In addition to the production of 3-chloro-2-hydroxyltrimethylammonium chloride, also known are several byproducts, such as 1,3-dichloro-2-propanol, 3-chloropropanediol, epoxy byproduct, and bis(trimethylammonium chloride)-2-propanol, i.e., the diquarternary salt.

U.S. Pat. No. 5,077,435 discloses a process to carry out the reaction of epichlorohydrin and trimethylamine hydrochloride in the presence of 1,3-dichloro-2-propanol as a cosolvent to reduce the formation of diquarternary salt, epoxy byproduct, and dichloropropanol. U.S. Pat. No. 5,463,127 further discloses that the reaction of epichlorohydrin and trimethylamine hydrochloride should be carried out at an initial pH of at least 8 by adding trimethylamine. U.S. Pat. No. 4,602,110 discloses a method to purify the product by crystallization from water-soluble alcohols.

U.S. Pat. No. 4,450,295 discloses a process of producing anhydrous trimethylamine chloride and then reacting it with epichlorohydrin to produce anhydrous 3-chloro-2-hydroxypropyl trimethylammonium chloride that is practically free of diquarternary bis(trimethylammonium chloride)-2-propanol.

U.S. Pat. No. 4,594,452 discloses a process for producing solid anhydrous 3-chloro-2-hydroxypropyl trimethylammonium chloride by carrying out the reaction of epichlorohydrin and trimethylamine hydrochloride in an organic solvent, which is a solvent for the two reactants, but a non-solvent for the product. Chloroform was disclosed to be a particularly suitable solvent.

JPH03287567 discloses in detail the reaction of chiral epichlorohydrin with trimethylamine hydrochloride to produce an optically active 3-chloro-2-hydroxylpropyl trimethylammonium chloride. When (S)-epichlorohydrin was reacted with an aqueous solution of trimethylamine hydrochloride, a solid product was obtained in a yield of 98.1% on the basis of trimethylamine. This crude product had a specific rotation of [α]_{D}²⁵=−23.4° (c=1.0, H₂O) and was purified by recrystallization twice in water to obtain a pure product, L-3-chloro-2-hydroxylpropyl trimethylammonium chloride, which has a specific rotation of [α]_{D}²⁵=−30.1° (c=1.0, H₂O) and a melting point of 215.5° C. When (R)-epichlorohydrin was used in the same reaction, a product was obtained in a yield of 96.8% with a specific rotation of [α]_{D}²⁵=+23.2° (c=1.0, H₂O). Recrystallization yielded a product of [α]_{D}²⁵=+29.5° (c=1.0, H₂O) and a melting point of 210.0-214.5° C. Despite the use of optically pure (S)- or (R)-epichlorohydrin in the reaction with trimethylamine hydrochloride, the product was not obtained as optically pure and the optical purity of the crude product was only about 78%. Hence, a partially racemized product was obtained even when optically pure (S)-epichlorohydrin was reacted with trimethylamine hydrochloride under the disclosed reaction conditions.

CN 102329243 discloses a continuous process in a tubular reactor for the reaction of (S)-epichlorohydrin dissolved in methanol with an aqueous solution of trimethylamine hydrochloride, wherein the ratio of the methanol solution of (S)-epichlorohydrin and the aqueous solution of trimethylamine hydrochloride is 1:0.92 (v/v). The product was obtained in a yield of 97% with a melting point of 190-191° C., which is much lower than 215° C. for a pure L-3-chloro-2-hydroxylpropyl trimethylammonium chloride. Hence, the method according CN 102329243 did not yield an optically pure product.

Since the reaction of L-3-chloro-2-hydroxylpropyl trimethylammonium chloride (II) with alkali cyanide to form L-carnitinenitrile chloride (III) and the subsequent hydrolysis of L-carnitinenitrile chloride to L-carnitine do not involve the chiral center, an optically impure L-3-chloro-2-hydroxylpropyl trimethylammonium chloride, produced according to prior art processes as an intermediated that is not isolated and purified, will result in the formation of an optically impure L-carnitinenitrile chloride and sodium chloride. In order to produce optically pure L-carnitine, it has been known to recrystallize L-carnitinenitrile chloride to enrich its optical purity and to separate sodium chloride from L-carnitinenitrile chloride. This isolation and purification process is complicated and the yield of L-carnitinenitrile chloride is significantly reduced. As a result, the yield of L-carnitine is greatly reduced.

It has been well known to produce L-carnitine from L-carnitinenitrile chloride by using an acid, in particular, hydrochloric acid, to hydrolyze the nitrile group to a carboxylic acid and ammonium chloride as a co-product. In order to isolate L-carnitine, hydrochloric acid in the solution is conventionally neutralized with ammonia or ammonium bicarbonate to form ammonium chloride. As a result, a solution of L-carnitine from a hydrolysis reaction contains about three moles of ammonium chloride for each mole of L-carnitine produced, since about 2 moles of hydrochloric acid are used in the hydrolysis of each mole of L-carnitinenitrile chloride. Therefore, L-carnitine has to be isolated from a solution containing a large amount of ammonium chloride. The desalting of L-carnitine from these large amounts of inorganic salts has represented one of the most difficult aspects in the industrial production of L-carnitine. Moreover, large amounts of waste water are generated during the desalting step by either ion exchange process or electrodialysis process. The disposal of these waste water containing high concentration of inorganic salts, particularly inorganic ammonium salts, is difficult and expensive, in order to comply with ever stringent environment protection requirement.

JPH 01287065A attempts to reduce the amounts of inorganic salts in the production of L-carnitine by converting L-carnitinenitrile chloride first with hydrogen peroxide to L-carnitinamide chloride in the presence of a base, followed by hydrolysis with sodium hydroxide. Although the process can reduce the formation of inorganic salts to a molar amount, the use of large amount of hydrogen peroxide and the generation of byproducts are still problematic. In addition, L-carnitine has been known to be unstable in a basic solution under the disclosed reaction conditions. As a result, the yield of L-carnitine produced according to this process is suboptimal.

It is an objective of the present invention to ameliorate these disadvantages and to disclose an improved process for the production of L-carnitine. It is another objective of the present invention to simplify the process by eliminating the isolation and purification of L-carnitinenitrile chloride. It is a further objective of the present invention to greatly reduce the amounts of inorganic salts produced in a solution of L-carnitine.

### SUMMARY OF THE INVENTION

The present invention discloses an improved process for the production of L-carnitine by eliminating the step of isolating L-carnitinenitrile chloride and by greatly reducing the formation of inorganic salts in a solution of L-carnitine. The invention is accomplished by removing an acid, especially hydrochloric acid, used in the hydrolysis of L-carnitinenitrile chloride from a solution of L-carnitine. The process according to the present invention can be carried out in a cascade of reactions from L-3-chloro-2-hydroxylpropyl trimethylammonium chloride to L-carnitine without isolating any intermediate.

### DETAILED DESCRIPTION OF THE INVENTION

The term “alkyl” refers to a straight, branched chain, or cyclic alkane (hydrocarbon) radical containing from 1 to 30 carbon atoms. Exemplary “alkyl” groups include methyl, ethyl, propyl, isopropyl, cyclopropyl, n-butyl, t-butyl, isobutyl, cyclobutyl, cyclopropylmethyl, pentyl, isopentyl, hexyl, isohexyl, heptyl, 4,4-dimethylpentyl, octyl, 2,2,4-trimethylpentyl, nonyl, decyl, undecyl, dodecyl, and the like. The term “C₁-C₃₀ alkyl” refers to a straight, cyclic, or branched chain alkane radical containing from 1 to 30 carbon atoms, such as methyl, ethyl, propyl, isopropyl, cyclopropyl, n-butyl, t-butyl, isobutyl, cyclobutyl, cyclopropylmethyl, pentyl, isopentyl, hexyl, isohexyl, heptyl, 4,4-dimethylpentyl, octyl, nonyl, decyl, undecyl, dodecyl, tridecyl, tetradecyl, pentadecyl, hexadecyl, hexydecyl or cetyl, heptadecyl, octadecyl, or stearyl, nonadecyl, eicodecyl, henicosyl, docosyl, tricosyl, tetracosyl, pentacosyl, hexacosyl, heptacosyl, octacosyl, nonacosyl, and tricontyl.

The compounds of the present invention may form salts which are also within the scope of this invention. Reference to compounds of the formula (I) through (IV) herein is understood to include reference to the salts thereof, unless otherwise indicated. The term “salt(s)”, as employed herein, denotes acidic and/or basic salts formed with inorganic and/or organic acids and bases.

The compounds of the present invention may form salts with a variety of organic and inorganic acids. Exemplary acid addition salts include hydrochloric acid, hydrobromic acid, sulfuric acid, nitric acid, phosphoric acid, alkanoic acids, alkylsulfonic acids, aromatic sulfonic acids, isethionic acid, and the like.

The compounds of the present invention may also form salts with a variety of organic and inorganic bases. Exemplary basic salts include ammonium salts, alkali metal salts such as lithium, sodium, potassium salts, alkaline earth metal salts such as calcium and magnesium salts, salts with organic bases (for example, organic amines) and salts with amino acids such as arginine, lysine, and the like.

All stereoisomers of the present compounds (for example, those which may exist due to asymmetric carbons on various substituents), including enantiomeric forms and diastereomeric forms, are contemplated within the scope of the invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The individual optical isomers can be obtained from the racemates by any suitable method, including, without limitation, conventional methods, such as salt formation with an optically active acid or base followed by crystallization, or biocatalytic methods, for example, selective hydrolysis with a lipase.

The present invention relates to an improved process for the production of L-carnitine, in particular, it relates to a process for the production of L-carnitine that can avoid the isolation and purification of L-carnitinenitrile chloride and it further relates to a process to reduce the amounts of inorganic salts produced in a solution of L-carnitine.

The invention is accomplished by a surprising and unexpected discovery that an L-carnitine salt (in particular, L-carnitine hydrochloride) can precipitate sodium chloride and ammonium chloride from a hydrolysis solution of L-carnitinenitrile chloride and optionally sodium chloride. The precipitated ammonium chloride and, optionally, sodium chloride can be readily separated from a solution of L-carnitine hydrochloride.

The invention is further accomplished by another surprising and unexpected discovery that an acid used in the hydrolysis reaction of L-carnitinenitrile chloride can be neutralized with an organic base to form a hydrophobic salt that can be separated either by phase separation or by solid-liquid separation to obtain a nearly neutral solution of L-carnitine. A solution of L-carnitine produced in the process according to the present invention contains much less than molar amounts of inorganic salts, even if an equal molar mixture of L-carnitinenitrile and sodium chloride is used in a hydrolysis reaction.

The process according to the present invention starts with the reaction of practically pure L-3-chloro-2-hydroxylpropyl trimethylammonium chloride with a source of cyanide to form L-carnitinenitrile chloride. A suitable source of cyanide is selected from the group consisting of alkali cyanide, alkaline earth metal cyanide, zinc cyanide, and a cyanohydrin; wherein the alkali is lithium, sodium, or potassium, and wherein the alkaline earth metal is magnesium, calcium, or barium. Preferably, an alkali cyanide is used; more preferably, sodium cyanide is used. When an alkali cyanide is used, the product is an aqueous solution of L-carnitinenitrile and alkali chloride.

A suitable material of L-3-chloro-2-hydroxylpropyl trimethylammonium chloride of formula (II) can be produced by any method or obtained from any source. It can be used as an isolated intermediate or as prepared in situ. Preferably, it is produced by a reaction of (S)-epichlorohydrin with trimethylamine hydrochloride in an organic solvent according to the process disclosed in the copending application Ser. No. 18/075,108, which is incorporated herein by reference.

The reaction of L-3-chloro-2-hydroxylpropyl trimethylammonium chloride with an alkali cyanide can be performed in an aqueous solution, optionally in the presence of water-soluble solvents. Suitable solvents are selected from the group consisting of methanol, ethanol, n-propanol, isopropanol, butanol, isobutanol, tert-butanol, 2-butanol, methoxyethanol, ethoxyethanol, propoxyethanol, ethylene glycol, diethylene glycol, propylene glycol, dimethylformamide, dimethylacetamide, dimethyl sulfone, N-methylpyrrolidinone, 1,3-dimethylimidazolidinone, tetramethylurea, and a mixture thereof.

The molar ratio of L-3-chloro-2-hydroxylpropyl trimethylammonium chloride and an alkali cyanide is from 1:2; preferably, 1:1.5; more preferably 1:1.1; most preferably, 1:1.05. A slight excess of alkali cyanide is used to ensure a complete conversion of L-3-chloro-2-hydroxylpropyl trimethylammonium chloride.

After the reaction of L-3-chloro-2-hydroxylpropyl trimethylammonium chloride with an alkali cyanide to form L-carnitinenitrile and alkali chloride, a small amount of excess alkali cyanide is still present in the solution. Moreover, the solution of L-carnitinenitrile chloride as produced is dark reddish. It is advantageous to remove this residual alkali cyanide, in order to ensure process safety and product safety.

Preferably, L-carnitinenitrile chloride is decolorized and residual cyanide is removed by using an oxidant, according to a process disclosed in the copending application Ser. No. 18/090,005, which is incorporated herein by reference. Suitable oxidants usable in the present invention are selected from the group consisting of hydrogen peroxide, urea hydrogen peroxide, performic acid, peracetic acid, perpropionic acid, perbenzoic acid, chloroperbenzoic acid, alkyl peroxide, dialkyl peroxide, benzoyl peroxide, chlorobenzoyl peroxide, alkali peroxide, alkaline earth metal peroxide, alkali percarbonate, alkali perborate, alkali hypochlorite, alkali hypobromite, alkaline earth metal hypochlorite, alkaline earth metal hypobromite, alkali persulfate, ammonium persulfate, N-chlorosuccinimide, N-bromosuccinimide, N-chlorophthalimide, N-bromophthalimide, 1,3-dichlorodimethylhydantoin, 1,3-dibromodimethylhydantoin, bromochlorodimethylhydantoin, trichloroisocyanuric acid, tribromoisocyanuric acid, alkali dichloroisocyanurate, alkali dibromoisocyanurate, alkali chloroisocyanurate, alkali bromoisocyanurate, and a mixture thereof, wherein the alkyl is a C₁-C₁₂ group, the alkali is lithium, sodium, potassium, or cesium, and the alkaline earth metal is magnesium, calcium, or barium.

Preferably, the oxidant is hydrogen peroxide or alkali hypochlorite. When hydrogen peroxide is used as the oxidant, water is the only byproduct left in an aqueous solution. Thus, no byproduct is introduced into the reaction solution. When alkali hypochlorite is used as the oxidant, alkali chloride is produced as a byproduct. However, this byproduct of alkali chloride is the same as the coproduct of L-carnitinenitrile chloride in a reaction of L-3-chloro-2-hydroxylpropyl trimethylammonium chloride and alkali cyanide. Thus, no different kind of byproduct is formed.

It has been found that residual cyanide in a solution of L-carnitinenitrile chloride can be removed by forming a stable adduct of cyanide. A suitable adduct has been found to be a cyanohydrin of aldehydes and ketones. Useful aldehydes and ketones are selected from the group consisting of, but not limited to, formaldehyde, acetaldehyde, propionaldehyde, glycolaldehyde, glyoxal, glyoxylic acid, acetone, butanone, cyclohexanone, cyclopentanone, pyruvic acid, glucose, galactose, fructose, xylose, sucrose, lactose, maltose, and a mixture thereof. Preferably, glyoxal, glyoxylic acid, pyruvic acid, cyclopentanone, or cyclohexanone is used. More preferably, glyoxylic acid is used.

When glyoxylic acid is used to neutralize a basic reaction solution of L-3-chloro-2-hydroxylpropyl trimethylammonium chloride with an alkali cyanide, the concentration of free cyanide falls to below 2 ppm. Preferably, the concentration of free cyanide is less than 1 ppm. More preferably, the concentration of free cyanide is less than 0.5 ppm. Most preferably, the concentration of free cyanide is less than 0.1 ppm.

After the removal of free cyanide from a solution of L-carnitinenitrile chloride and alkali chloride, L-carnitinenitrile chloride may be isolated from this solution by methods known to one skilled in the art. The separation of L-carnitinenitrile chloride from alkali chloride can be conventionally achieved by using a lower alcohol, for instance, methanol or ethanol. The isolated L-carnitinenitrile chloride is then mixed with an acid to carry out a hydrolysis of L-carnitinenitrile chloride to form L-carnitine and ammonium chloride.

It has been found that L-carnitinenitrile chloride thus produced can be used to produce L-carnitine without further purification, if L-3-chloro-2-hydroxylpropyl trimethylammonium chloride of formula (II) used in the reaction is practically optically pure. It has further been found that alkali chloride, produced along with L-carnitinenitrile chloride, does not need to be separated from L-carnitinenitrile chloride, in the production of L-carnitine in the process according to the present invention.

Preferably, a solution of L-carnitinenitrile chloride and alkali chloride can be concentrated to crystallize L-carnitinenitrile chloride and alkali chloride without separation. It is surprising and unexpected to find that L-carnitinenitrile chloride can be obtained as nearly white solid, if it is crystallized from this aqueous solution. L-carnitinenitrile chloride and alkali chloride can be recovered from the solution in a yield of greater than 90%; preferably, greater than 95% in a cyclic process, wherein the mother liquor solution can be further concentrated to yield additional product or combined with a fresh solution of L-carnitinenitrile chloride and alkali chloride.

This solid mixture of L-carnitinenitrile chloride and alkali chloride can be separated into L-carnitinenitrile chloride and alkali chloride by any method known to one skilled in the art. L-carnitinenitrile chloride is then hydrolyzed with an acid to L-carnitine and ammonium chloride. A suitable acid is selected from the group consisting of hydrochloric acid, hydrobromic acid, sulfuric acid, nitric acid, alkyl sulfonic acid, aryl sulfonic acid, and a mixture thereof. Preferably, the acid is hydrochloric acid.

Preferably, it has been found that a solid mixture of L-carnitinenitrile chloride and alkali chloride can be mixed with an acid to carry out a hydrolysis reaction to form L-carnitine. More preferably, a reaction solution of L-carnitinenitrile chloride and alkali chloride is mixed with an acid to carry out a hydrolysis reaction to form L-carnitine. A suitable acid is selected from the group consisting of hydrochloric acid, hydrobromic acid, sulfuric acid, nitric acid, alkyl sulfonic acid, aryl sulfonic acid, and a mixture thereof. Preferably, the acid is hydrochloric acid.

When hydrochloric acid is used in the hydrolysis reaction, the product solution is comprised of excess hydrochloric acid, L-carnitine hydrochloride, ammonium chloride, and optionally alkali chloride. It has been surprising and unexpected to find that L-carnitine hydrochloride can precipitate ammonium chloride and alkali chloride from a solution upon cooling. This discovery renders prior separation of L-carnitinenitrile chloride and alkali chloride unnecessary, as both ammonium chloride and alkali chloride can be removed from the solution at the same time. The precipitated solids of ammonium chloride and alkali chloride can be separated by any method known to one skilled in the art. The amounts of precipitated salts are more than 50% of the molar amount of ammonium chloride and alkali chloride formed in the reactions. Preferably, more than 60% of the amounts of salts is removed. More preferably, more than 70% of the salts are removed. Most preferably, more than 80% of the salts are removed to obtain a solution of L-carnitine in the presence of excess hydrochloric acid and a small amount of residual inorganic salts of ammonium chloride and alkali chloride.

After the removal of precipitated ammonium chloride and alkali chloride, L-carnitine can be isolated from the mother liquor solution by any method known to one skilled in the art. For example, the solution can be neutralized with a base and a neutralized solution of L-carnitine is subject to an ion exchange resin process or an electrodialysis to isolate a salt-free L-carnitine. A suitable base is selected from the group consisting of ammonia, ammonium bicarbonate, ammonium carbonate, alkali hydroxide, alkali bicarbonate, alkali carbonate, and a mixture thereof. However, these conventional methods of neutralization are disadvantageous as more salts are formed in a solution of L-carnitine.

It has now been found that an acid and L-carnitine can be separated from each other by using an organic base, whose salt of the acid can be separated from the aqueous solution either by solid-liquid separation or by phase separation. The neutralization in the process according to the present invention is optionally carried out in the presence of a water-insoluble solvent. This discovery is particularly surprising since it is unexpected that L-carnitine is not removed along with the acid salt of the organic base, despite the fact that L-carnitine itself may from a salt with an organic base.

The process according to the present invention to separate an acid and L-carnitine is applicable to an acid solution of L-carnitine from any source or produced by any method. The process according to the present invention is particularly suitable for an acid hydrolysis solution of L-carnitinenitrile chloride and optionally in the presence of alkali chloride.

One class of suitable organic bases is an amine of the formula: NR¹R²R³, wherein R¹, R², and R³, is each independently hydrogen or a C₁-C₃₀ alkyl group, and wherein the total number of carbons of R¹, R², and R³ is at least 12, preferably, at least 14, more preferably, at least 16.

Suitable amines for instance are selected from the group consisting of, but not limited to, tributylamine, tripentylamine, trihexylamine, trioctylamine, tri(2-ethylhexyl)amine, dicyclohexylamine, dibenzylamine, tribenzylamine, triisooctylamine, trilaurylamine, methyl di-n-octylamine, dilauryl amine, dibutyl-n-dodecylamine, dibutyl-n-decylamine, and di-isobutyl-n-octylamine.

Suitable amines are also selected from the group consisting of commercially available mixed amines: Amberlite LA-1, Amberlite LA-2, Primene JM, Primene JMT, Primene 81R, Amin 90-178, Amine 21F-81, Aminsco, Arquad 2C, Amine-24, B-104, Amberlite NE-204, and N235. Suitable amines are further selected from the group consisting of alkyl substituted azacycloalkanes of the following formula:

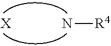

wherein X is (CH₂)ₘ, (CH₂)ₘO(CH₂)ₙ, (CH₂)ₘS(CH₂)ₙ, or acyl-N(CH₂)ₘ(CH₂)ₙ; wherein m and n is each an integer from 4 to 12, R⁴ is an alkyl group, acyl is an alkyl or aryl acyl group, and wherein the methylene group may be substituted with an alkyl group in the total sum of alkyl groups. The number of total carbons of methylene groups and R⁴ is at least 14.

Another class of suitable organic bases belongs to an imidazole derivative of the formula:

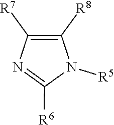

wherein R⁵, R⁶, R⁷, and R⁸ is each independently hydrogen or an alkyl group and wherein the number of total carbons of R⁵ to R⁸ is at least 10, preferably, at least 12, more preferably, at least 14.

A further class of suitable organic bases belongs to a pyridine derivative of the formula:

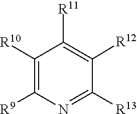

wherein R⁹, R¹⁰, R¹¹, R¹², and R¹³ is each independently hydrogen, alkyl group, acylamino group, alkylsulfonylamino group, or arylsulfonylamino group and wherein the number of total carbons of R⁹, R¹⁰, R¹¹, R¹², and R¹³ is at least 8, preferably, at least 10, more preferably, at least 12.

It is also possible that other organic bases not defined above are applicable. Therefore, the organic bases cited should be viewed as typical but neither as optimal nor restrictive.

A solvent used in conjunction with an organic base in the process according to the present invention can be any solvent or mixed solvent that is substantially insoluble in water and not reactive towards the organic base. Suitable solvents are selected from the group consisting of alcohols of at least C₄ alkyl group, esters, ethers, ketones, aliphatics, aromatics, amides of at least C₈ alkyl group, and mixture thereof. For instance, suitable solvents are selected from, but not limited to, butanol, isobutanol, 2-butanol, amyl alcohol, isoamyl alcohol, hexanol, iso-hexanol, cyclohexanol, octanol, iso-octanol, 2-octanol, 2-ethylhexanol, decanol, dodecanol, butyl formate, ethyl acetate, butyl acetate, butanone, methyl isobutyl ketone, cyclohexanone, pentane, hexane, cyclohexane, heptane, octane, dodecanes, kerosene, diethyl ether, dibutyl ether, dipropyl ether, diisopropyl ether, benzene, toluene, xylene, cumene, chlorobenzene, dichloromethane, dichloroethane, chloroform, trichloroethylene, tetrachloroethylene, dibutyl acetamide, dihexyl acetamide, dibutyl formamide, dibutyl acetamide, dihexyl acetamide, and dihexyl formamide. Preferably, kerosene is used.

The process according to the present invention is carried out by mixing an organic base, optionally a solvent, with an acid solution of L-carnitine. After mixing, the mixture is settled for phase separation. It has been found that the phase separation can be facilitated by the use of a centrifugal phase separator, if the phase separation becomes difficult. After phase separation, the aqueous phase contains L-carnitine, while the organic phase or phases contains the acid salt of the organic base. It has been seen that two organic phases are observed in the absence of a solvent. One organic phase is the free base if used in more than a molar amount of the acid. The other organic phase is the hydrochloride salt. It is surprising and unexpected that L-carnitine is not found in either of the two organic phases.

In a few instances of organic bases, for example, dicyclohexylamine, dibenzylamine, and N-benzyl-N-cyclohexylamine, their hydrochloric acid salts are insoluble in water and precipitate as a solid. These solid salts can be separated by any means of solid-liquid separation techniques, such as filtration, centrifuge, or press filtration.

In the neutralization reaction in the process according to the present invention, the amount of organic base is not limited. Preferably, the amount is at least equal to the molar amount of acid present in an acid solution of L-carnitine. A lesser amount of organic base may be used, but the solution after treatment remains strongly acidic. Excess amount of organic base may be used, but no advantage is gained. If at least a molar amount of organic base relative to the acid is used in the neutralization, the L-carnitine solution is found to be in a pH range of 5-6.

A free base form of organic base can be readily regenerated from an acid salt by reacting the acid salt, optionally in the presence of a solvent, with an aqueous solution of ammonia or with a solution of alkali hydroxide or alkali carbonate. The amount of aqueous ammonia or an alkali hydroxide used in the regeneration is not limited. Preferably, the amount is at least equal to the molar amount of the acid in an acid salt. After regeneration, an organic base remains in the organic phase, while an ammonium or alkali salt of the acid stays in the aqueous phase. The aqueous phase can be concentrated to recover the salt. Excess ammonia may be recovered for reuse during the concentration. Excess alkali hydroxide may be recycled to the regeneration stage after the salt is crystallized and separated. The organic base in the solvent can be used repetitively in the neutralization reaction with little loss.

After the removal of an acid, a nearly neutral solution of L-carnitine can be obtained. Preferably, the solution of L-carnitine has a range of pH from 5 to 7. A solution of L-carnitine produced in the process according to the present invention contains much less than molar amounts of inorganic salts on the basis of L-carnitine. In fact, the content of inorganic salts is less than 10% relative to L-carnitine by weight. The amounts of inorganic salts in a solution of L-carnitine produced in the process according to the present invention are nearly an order less than conventional process wherein the weight of inorganic salts is about the same as the weight of L-carnitine.

L-carnitine can be isolated and purified from this aqueous solution by methods known to one skilled in the art, for example, by using an ion exchange resin or by using electrodialysis. Since the amounts of inorganic salts in the solution of L-carnitine produced according to the invention are much reduced, the productivity is greatly enhanced. Most importantly, product loss in the desalting step of the process using ion exchange resin or electrodialysis can be greatly reduced.

The process according to the present invention can be carried out in stages starting from a reaction of L-3-chloro-2-hydroxypropyl trimethylammonium chloride of formula (II) with alkali cyanide to form L-carnitinenitrile chloride and alkali chloride. After L-carnitinenitrile is isolated from its mixture with alkali chloride and purified, L-carnitinenitrile chloride is then converted to L-carnitine. Preferably, the process according to the present invention can be carried out in a cascade of reactions from the starting material to the final product of L-carnitine without isolating any intermediate. Hence, the process according to the present invention can greatly simplify the production process of L-carnitine.

After L-carnitine is isolated, the L-carnitine can be converted to L-carnitine L-tartrate by reacting with L-tartaric acid, L-carnitine fumarate with fumaric acid, and acetyl-L-carnitine hydrochloride with acetyl chloride, propionyl-L-carnitine hydrochloride with propionyl chloride, by processes known in the art.

The process according to the present invention can be carried out discontinuously, semi-continuously, or continuously.

### EXAMPLES

The following examples will illustrate the practice of this invention but are not intended to limit its scope.

### Example 1

To a round-bottom flask were added 100 mL of 30% hydrochloric acid and 90 g of L-carnitinenitrile chloride. After the clear solution was stirred and heated to 90-95° C. for four hours, the solution was cooled to 0-5° C. to precipitate ammonium chloride. The ammonium chloride was filtered and the filtration cake was washed with a little cold 30% hydrochloric acid to obtain a solution of L-carnitine hydrochloride, which was diluted with deionized water to 500 mL to prepare a stock solution of L-carnitine hydrochloride in hydrochloric acid.

### Example 2

50 mL of the stock solution of Example 1 was mixed well with 50 mL of trioctylamine in a separatory funnel. After settling, the lower aqueous phase of L-carnitine solution was separated. It showed a pH of 5. Two organic phases were observed. The two phases were washed with 50 mL of 0.5 M hydrochloric acid. After settling, the aqueous solution was separated and used to measure optical rotation. The measurement did not show any optical rotation.

### Example 3

50 mL of the stock solution of Example 1 was mixed well with 50 mL of tris(2-ethylhexyl)amine in a separatory funnel. After settling, the lower aqueous phase of L-carnitine solution was separated. It showed a pH of 5. The upper organic phase was washed with 50 mL of 0.5 M hydrochloric acid. After settling, the aqueous solution was separated and used to measure optical rotation. The measurement did not show any optical rotation.

### Example 4

50 mL of the stock solution of Example 1 was mixed well with 25 mL of tris(2-ethylhexyl)amine and 25 mL of trioctylamine in a separatory funnel. After settling, the lower aqueous phase of L-carnitine solution was separated. It showed a pH of 5. The upper organic phase was washed with 50 mL of 0.5 M hydrochloric acid. After settling, the aqueous solution was separated and used to measure optical rotation. The measurement did not show any optical rotation.

### Example 5

50 mL of the stock solution of Example 1 was mixed well with 50 mL of trioctylamine and 50 mL of kerosene in a separatory funnel. After settling, the lower aqueous phase of L-carnitine solution was separated. It showed a pH of 5. The upper organic phase was washed with 50 mL of 0.5 M hydrochloric acid. After settling, the aqueous solution was separated and used to measure optical rotation. The measurement did not show any optical rotation.

### Example 6

50 mL of the stock solution of Example 1 was mixed well with 50 mL of trioctylamine, 50 mL of kerosene, and 10 mL of decanol in a separatory funnel. After settling, the lower aqueous phase of L-carnitine solution was separated. It showed a pH of 5. The upper organic phase was washed with 50 mL of 0.5 M hydrochloric acid. After settling, the aqueous solution was separated and used to measure optical rotation. The measurement did not show any optical rotation.

### Example 7

To a round-bottom flask were added 200 mL of water, 32.3 g of 2-ethylhexylamine, 21.0 g of ammonium bicarbonate, and 72.5 g of 40% glyoxal. The solution was stirred while 45 mL of butyraldehyde was added dropwise. The reaction was exothermic and the temperature was maintained at 35-45° C. for 2 hours. The upper oil phase was separated and distilled under vacuum to obtain a yellowish oil of N-(2-ethylhexyl)-2-propylimidazole.

50 mL of the stock solution of Example 1 was mixed well with 50 g of N-(2-ethylhexyl)-2-propylimidazole and 50 mL of kerosene in a separatory funnel. After settling, the lower aqueous phase of L-carnitine solution was separated. It showed a pH of 5. The upper organic phase was washed with 50 mL of 0.5 M hydrochloric acid. After settling, the aqueous solution was separated and used to measure optical rotation. The measurement did not show any optical rotation.

### Example 8

To a round-bottom flask were added 150 mL of piperidine and 102 g of dodecyl chloride. After the solution was refluxed for 2 hours, excess piperidine was distilled off. To the residue was added 200 ml of water containing 40 g of sodium hydroxide. The oil phase was isolated as N-dodecylpiperidine.

50 mL of the stock solution of Example 1 was mixed well with 50 g of N-dodecylpiperidine and 50 mL of kerosene in a separatory funnel. After settling, the lower aqueous phase of L-carnitine solution was separated. It showed a pH of 5. The upper organic phase was washed with 50 mL of 0.5 M hydrochloric acid. After settling, the aqueous solution was separated and used to measure optical rotation. The measurement did not show any optical rotation.

### Example 9

The organic phase of 50 g of trioctylamine, kerosene, and 10 g of decanol used in Example 6 was washed with 100 mL of 1 N sodium hydroxide to regenerate the free base. After separating off the lower aqueous phase, 50 mL of the stock solution of Example 1 was added and mixed well. After settling, the lower aqueous phase of L-carnitine solution was separated. It showed a pH of 5. The upper organic phase was washed with 50 mL of 0.5 M hydrochloric acid. After settling, the aqueous solution was separated and used to measure optical rotation. The measurement did not show any optical rotation.

### Example 10

To a round bottom flask was added 50 mL of the stock solution of Example 1. The solution was stirred while 18.8 g of dicyclohexylamine was added dropwise to form crystalline solid of dicyclohexylamine hydrochloride. After the precipitate was filtered and washed with deionized water, the mother liquor solution of L-carnitine showed a pH of 6.

### Example 11

To a round bottom flask was added 50 mL of the stock solution of Example 1. The solution was stirred while 20.1 g of dicyclohexylamine was added dropwise to form crystalline solid of dibenzylamine hydrochloride. After the precipitate was filtered and washed with deionized water, the mother liquor solution of L-carnitine showed a pH of 6.

### Example 12

To a round bottom flask were added 94 g of L-3-chloro-2-hydroxypropyl trimethylammonium chloride (0.5 mol) and 100 mL water. After the solution was stirred and warmed to 35° C., a solution containing 25.5 g of sodium cyanide was added in three portions over a period of about 1 hr. The temperature was maintained between 35° C. and 45° C. for 5 hours. The reaction solution changed from nearly colorless at the beginning to dark reddish and opaque. Afterwards, 4.5 mL of 35% hydrogen peroxide was added to the flask and the temperature was kept at 45° C. for about 1 hr. The dark reddish solution became a light yellowish and clear solution. A cyanide test showed 0.2 ppm of free cyanide.

To a round bottom flask were added 282 g of L-3-chloro-2-hydroxypropyl trimethylammonium chloride (1.5 mol) and 100 mL water to obtain a suspension. After the suspension was stirred and warmed to 35° C., 250 mL of a solution containing 75.6 g of sodium cyanide (1.54 mol) was added in three portions over a period of about 1 hr. The temperature was maintained between 35° C. and 45° C. for 5 hours. The reaction solution changed from nearly colorless at the beginning to dark reddish and opaque. Afterwards, 13 mL of 35% hydrogen peroxide was added to the flask and the temperature was kept at 45° C. for about 1 hr, then at 65° C. for 1 hr. The dark reddish solution became a light yellowish and clear solution. Free cyanide in the solution could not be detected (<0.1 ppm).

The reaction solutions were combined and concentrated to a crystalline suspension. The suspension was then cooled to room temperature and filtered to obtain a crystalline mixture of L-carnitinenitrile chloride and sodium chloride. The mother liquor solution was repeatedly concentrated and filtered to obtain a crystalline mixture of L-carnitinenitrile chloride and sodium chloride. After drying, the solid weighted 452 g in a molar yield of 96%.

To a round-bottom flask were added 100 mL of 30% hydrochloric acid and 100 g of a solid mixture of L-carnitinenitrile chloride and sodium chloride. After the suspension was stirred and heated at 95° C. on a water bath for 4 hours, the suspension was cooled to 5-10° C. to precipitate sodium chloride and ammonium chloride. The suspension was filtered and washed with a little cold 30% hydrochloric acid. After drying, the solid weighted 41.5 g.

The filtration solution was diluted with deionized water to about 300 mL, transferred to a separatory funnel, and mixed well with 500 mL of trioctylamine and kerosene prepared from 300 mL of trioctylamine and 200 mL of kerosene. The organic phase was then regenerated with 500 mL of 10% sodium hydroxide and mixed with the L-carnitine solution. After two treatments, the aqueous solution of L-carnitine showed a pH of 5.

The aqueous phase was divided into two equal portions and applied to about 500 mL of a strongly acidic resin bed. After washing with deionized water to a neutral eluent, the absorbed L-carnitine was eluted with an aqueous solution of 3% ammonia. The L-carnitine fractions were combined and evaporated to dryness. The residue was dissolved in a minimal amount of anhydrous ethanol and L-carnitine was precipitated with acetone. After filtration, washing with acetone, and drying, the white crystalline product weighted 59.1 g. [α]_{D}²⁵=−30.2° (c=10, H₂O).

It will be understood that the foregoing examples and explanation are for illustrative purposes only and that various modifications of the present invention will be self-evident to those skilled in the art. Such modifications are to be included within the spirit and purview of this application and the scope of the appended claims.

What is claimed is:

1. A process for the production of L-carnitine, comprising:
(a) mixing an organic base with an acid solution comprised of L-carnitine to form a salt of the organic base and the acid, optionally in the presence of a solvent;
(b) removing the salt of step (a) to yield a solution comprised of L-carnitine; and
(c) isolating the L-carnitine from the solution of step (b).

2. The process according to claim 1, wherein the acid is selected from the group consisting of hydrochloric acid, hydrobromic acid, sulfuric acid, nitric acid, phosphoric acid, alkylsulfonic acid, arylsulfonic acid, and a mixture thereof.

3. The process according to claim 1, wherein the acid is hydrochloric acid.

4. The process according to claim 1, wherein the acid solution of L-carnitine is produced by an acid hydrolysis of L-carnitinenitrile chloride.

5. The process according to claim 1, wherein the acid solution of L-carnitine is produced by an acid hydrolysis of L-carnitinenitrile chloride and alkali chloride.

6. The process according to claim 1, wherein the organic base is an amine of the formula: NR¹R²R³, wherein R¹, R², and R³ is each independently hydrogen or C₁-C₂₄ alkyl group, and wherein the number of total carbons of R¹, R², and R³ is at least 12.

7. The process according to claim 1, wherein the organic base is an alkyl azacycloalkane of the structure: 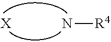
wherein X is (CH₂)ₘ, (CH₂)ₘO(CH₂)ₙ, (CH₂)ₘS(CH₂)ₙ, or acyl-N(CH₂)ₘ(CH₂)ₙ; wherein m and n is each an integer from 4 to 12, R⁴ is an alkyl group, acyl is an alkyl or aryl acyl group; wherein the methylene group may be substituted with an alkyl group in the total sum of alkyl groups; and the number of total carbons of methylene groups and R⁴ is at least 14.

8. The process according to claim 1, wherein the organic base is an imidazole derivative of the structure: 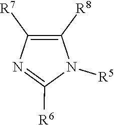
wherein R⁵, R⁶, R⁷, and R⁸ is each independently hydrogen or an alkyl group and wherein the total number of carbons of R⁵, R⁶, R⁷, and R⁸ is at least 10.

9. The process according to claim 1, wherein the organic base is a pyridine derivative of the structure: 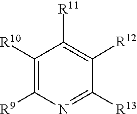
wherein R⁹, R¹⁰, R¹¹, R¹², and R¹³ is each independently hydrogen, alkyl group, acylamino group, alkylsulfonylamino group, or arylsulfonylamino group and wherein the number of total carbons of R⁹, R¹⁰, R¹¹, R¹², and R¹³ is at least 8.

10. The process according to claim 1, wherein the solvent is selected from the group consisting of alcohols of at least C₄ alkyl group, esters, ethers, ketones, aliphatics, aromatics, amides of at least eight total carbon alkyl groups, and a mixture thereof.

11. The process according to claim 1, wherein the salt is removed from the solution of L-carnitine by a phase separation or a solid-liquid separation.

12. The process according to claim 1, further comprising the production of L-carnitine L-tartrate by reacting the L-carnitine with L-tartaric acid.

13. The process according to claim 1, further comprising the production of L-carnitine fumarate by reacting the L-carnitine with fumaric acid.

14. The process according to claim 1, further comprising the production of acetyl-L-carnitine hydrochloride by reacting the L-carnitine with acetyl chloride.

15. The process according to claim 1, further comprising the production of propionyl-L-carnitine hydrochloride by reacting the L-carnitine with propionyl chloride.

16. A process for the production of L-carnitine, comprising:
(a) mixing L-carnitinenitrile chloride and optionally alkali chloride with hydrochloric acid to form a solution of L-carnitine hydrochloride, ammonium chloride, and, optionally, alkali chloride;
(b) cooling the solution of step (a) to precipitate the ammonium chloride and, optionally, alkali chloride;
(c) separating the precipitated ammonium chloride and, optionally, alkali chloride of step (b) to yield a solution of L-carnitine hydrochloride; and
(d) isolating the L-carnitine from the solution of step (c).

17. The process according to claim 16, further comprising a step of neutralizing the solution of L-carnitine hydrochloride with an organic base.
